(19) United States
(12) Patent Application Publication
(10) Pub. No.: US 2007/0298098 A1
(43) Pub. Date: Dec. 27, 2007

(54) Controlled Release Compositions Comprising Levetiracetam

(75) Inventors: Scott A. Jenkins, Downington, PA (US); Gary Liversidge, West Chester, PA (US)

Correspondence Address: SYNNESTVEDT & LECHNER LLP, 2600 ARAMARK TOWER, 1101 MARKET STREET, PHILADELPHIA, PA, 19107, US

(73) Assignee: ELAN PHARMA INTERNATIONAL LIMITED, Athlone (IE)

(21) Appl. No.: 11/568,841

(22) PCT Filed: Feb. 14, 2006
(86) PCT No.: PCT/US06/05167

§ 371 Date: Jun. 29, 2007

Related U.S. Application Data
(60) Provisional application No. 60/653,564, filed on Feb. 16, 2005.

(51) Int. Cl. A61K 9/22; A61K 31/40; A61K 9/10; A61P 25/08; A61K 9/48; A61K 9/14

(52) U.S. Cl. 424/456; 424/468; 424/484; 424/490; 514/424

(57) ABSTRACT

The invention relates to a controlled release composition comprising levetiracetam for the treatment of epilepsy. The controlled release composition comprises an immediate release component and a modified release component or formulation. The immediate release component comprises a first population of levetiracetam and the modified release component or formulation preferably comprises a second population of levetiracetam and a controlled release constituent. The modified release formulation is preferably in the form of an erodable formulation, a diffusion controlled formulation or an osmotic controlled formulation. The combination of the immediate release component and the modified release component or formulation in operation deliver the active ingredient in a pulsed or bimodal manner.

### FIELD OF INVENTION

The present invention relates to controlled release compositions comprising levetiracetam that are used preferably for the treatment of epilepsy.

### BACKGROUND OF INVENTION

Background Regarding Levetiracetam

Levetiracetam is an antiepileptic drug marketed under the registered trademark Keppra® by UCB Pharma, Inc., of Smyrna, Ga. Levetiracetam is available as 250 mg (blue), 500 mg (yellow) and 750 mg (orange) tablets and as a clear, colorless, grape-flavored liquid (100 mg/mL) for oral administration. The Physicians Desk Reference, 58^{th} Edition (2004), pp. 17, 337, 3230.

The chemical name of levetiracetam, a single enantiomer, is (−)(S)-α-ethyl-2-oxo-1-pyrrolidine acetamide, its molecular formula is C₈H₁₄N₂O₂ and its molecular weight is 170.21. Levetiracetam is chemically unrelated to existing antiepileptic drugs (AEDs).

Levetiracetam is a white to off-white crystalline powder with a faint odor and a bitter taste. It is very soluble in water (104.0 g/100 mL). It is freely soluble in chloroform (65.3 g/100 mL) and in methanol (53.6 g/100 mL), soluble in ethanol (16.5 g/100 mL), sparingly soluble in acetonitrile (5.7 g/100 mL) and practically insoluble in n-hexane. (Solubility limits are expressed as g/100 mL solvent.)

Keppra® tablets contain the labeled amount of levetiracetam. Inactive ingredients in the tablets are colloidal silicon dioxide, corn starch, hydroxypropyl methylcellulose, magnesium stearate, polyethylene glycol 4000, povidone, talc, titanium dioxide and coloring agents.

The individual tablets contain the following coloring agents:

| | |
|---|---|
| 250 mg tablets: | FD&C Blue No. 2, |
| 500 mg tablets: | yellow iron oxide. |

The pharmacokinetics of levetiracetam have been studied in healthy adult subjects, adults and pediatric patients with epilepsy, elderly subjects and subjects with renal and hepatic impairment.

Levetiracetam is rapidly and almost completely absorbed after oral administration. Levetiracetam tablets and oral solution are bioequivalent. The pharmokinetics are linear and time-invariant, with low intra- and inter-subject variability. The extent of bioavailability of levetiracetam is not affected by food. Levetiracetam is not protein-bound (<10% bound) and its volume of distribution is close to the volume of intracellular and extracellular water. Sixty-six percent (66%) of the dose is renally excreted unchanged. The major metabolic pathway of levetiracetam (24% of dose) is an enzymatic hydrolysis of the acetamide group. It is not liver cytochrome P450 dependent. The metabolites have no known pharmacological activity and are renally excreted. Plasma half-life of levetiracetam across studies is approximately 6-8 hours. It is increased in the elderly (primarily due to impaired renal clearance) and in subjects with renal impairment.

Absorption of levetiracetam is rapid, with peak plasma concentrations occurring in about an hour following oral administration in fasted subjects. The oral bioavailability of levetiracetam tablets is 100% and the tablets and oral solution are bioequivalent in rate and extent of absorption. Food does not affect the extent of absorption of levetiracetam but it decreases Cₘₐₓ by 20% and delays Tₘₐₓ by 1.5 hours. The pharmokinetics of levetiracetam are linear over the dose range of 500-5000 mg. Steady state is achieved after 2 days of multiple twice-daily dosing. Levetiracetam and its major metabolite are less than 10% bound to plasma proteins; clinically significant interactions with other drugs through competition for protein binding sites are therefore unlikely. The Physicians Desk Reference, 58^{th} Edition (2004), p. 3230.

Due to levetiracetam's high degree of bioavailability and rapid metabolism, it would be advantageous to provide nanoparticulate levetiracetam with a drug-delivery formulation that releases the active in a controlled or delayed release profile. More specifically, it would be a tremendous benefit to patients suffering from epilepsy if the drug could be formulated to be released in a two-phase or pulsatile manner so that the drug can provide its pharmacological activity over an extended period of time, in particular, a twenty-four hour period, instead of being rapidly metabolized. In this manner, patients suffering from epilepsy can benefit from the drug's therapeutic effects for extended periods of time.

Pertinent patents are U.S. Pat. No. 4,837,223 to Gobert et al., which is for “(S)-Alpha-Ethyl-2-Oxo-1-Pyrrolidineacetamide compositions” and U.S. Pat. No. 4,943,639, also to Gobert et al., which is for “(S)-Alphyl-Ethyl-2-Oxo-1-Pyrrolidineacetamide.” These patents are incorporated by reference herein.

### SUMMARY OF THE INVENTION

It is an object of the present invention to provide a controlled release composition containing levetiracetam, which in operation produces a plasma profile substantially similar to the plasma profile produced by the administration of two or more levetiracetam dosage forms given sequentially.

It is a further object of the invention to provide a controlled release composition, which in operation delivers levetiracetam in a pulsatile manner.

Another object of the invention is to provide a controlled release composition which substantially mimics the pharmacological and therapeutic effects produced by the administration of two or more immediate release dosage forms given sequentially.

Another object of the present invention is to provide a controlled release composition which substantially reduces or eliminates the development of patient tolerance to the levetiracetam of the composition.

Another object of the invention is to provide a controlled release composition in which a first portion of the levetiracetam is released immediately upon administration and a second portion of the levetiracetam is released rapidly after an initial delay period in a bimodal manner.

Another object of the present invention is to formulate the dosage in the form of erodable formulations, diffusion controlled formulations or osmotic controlled formulations.

Another object of the invention is to provide a controlled release composition capable of releasing the levetiracetam in a bimodal or multi-modal manner in which a first portion of the active is released either immediately or after a delay time to provide a pulse of drug release, and one or more additional portions of the levetiracetam is released, each after a respective lag time, to provide additional pulses of drug release during a period of up to twenty-four hours.

Another object of the invention is to provide solid oral dosage forms comprising a controlled release composition of the present invention, comprising levetiracetam.

Other objects of the invention include provision of a once daily dosage form of levetiracetam which, in operation, produces a plasma profile substantially similar to the plasma profile produced by the conventional administration of two immediate release dosage forms given sequentially and a method for treatment of epilepsy based on the administration of such a dosage form.

The present invention utilizes controlled release delivery of levetiracetam from a solid oral dosage formulation to allow dosage less frequently than before, and preferably once-a-day administration, increasing patient convenience and compliance. The mechanism of controlled release would preferably utilize, but not be limited to, erodable formulations, diffusion controlled formulations and osmotic controlled formulations. A portion of the total dose may be released immediately to allow for rapid onset of effect. The invention would be useful in improving compliance and, therefore, therapeutic outcome for all treatments requiring levetiracetam, including but not limited to, adjunctive therapy in the treatment of partial onset seizures in adults with epilepsy. This approach would replace conventional levetiracetam tablets and solution, which are administered twice a day as adjunctive therapy in the treatment of partial onset seizures in adults with epilepsy.

The present invention, therefore, relates to a controlled modified release composition for the controlled release of levetiracetam. In particular, the present invention relates to a controlled release composition that in operation delivers levetiracetam in a pulsatile manner, preferably during a period of up to twenty-four hours. The present invention further relates to solid oral dosage forms containing a controlled release composition.

The above objects are realized by a controlled release composition having a first component comprising a first population of levetiracetam particles and a second component or formulation comprising a second population of levetiracetam particles. The ingredient-containing particles of the second component further comprises a modified release constituent comprising a release coating or release matrix material, or both. Following oral delivery, the composition in operation delivers the levetiracetam in a pulsatile manner.

Preferred controlled release formulations are erodable formulations, diffusion controlled formulations and osmotic controlled formulations. According to the invention, a portion of the total dose may be released immediately to allow for rapid onset of effect, with the remaining portion of the total dose released over an extended time period. The invention would be useful in improving compliance and, therefore, therapeutic outcome for all treatments requiring levetiracetam, including but not limited to, the treatment of epilepsy.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

Controlled release compositions similar to those disclosed herein are disclosed and claimed in the U.S. Pat. Nos. 6,228,398 and 6,730,325 to Devane et al., both of which are incorporated by reference herein.

In a preferred embodiment of a multiparticulate modified release composition according to the invention the first component includes an immediate release constituent.

The modified release coating, when applied to the second population of levetiracetam particles, causes a lag time between the release of active from the first population of active levetiracetam containing particles and the release of active from the second population of active levetiracetam containing particles. The modified release matrix material, when applied to a second population of active levetiracetam particles, causes a lag time between the release of levetiracetam from the first population of levetiracetam particles and the release of active ingredient from the second population of active ingredient containing particles. The duration of the lag time may be varied by altering the composition and/or the amount of the modified release coating and/or altering the composition and/or amount of modified release matrix material utilized in the composition or formulation. Preferred types of formulations for use in varying the lag time are erodable formulations, diffusion controlled formulations and osmotic controlled formulations. Thus, the duration of the lag time can be designed to mimic a desired plasma profile.

Erodable Formulations

The subsequent formulations can be in the form of erodable formulations in which the active ingredients and modified release constituent consisting of at least one of modified release coatings and modified release matrix materials would dissolve in water, over time losing their structural integrity. One manner in which this could occur would be that the active ingredients and modified release coatings and/or matrix materials would dissolve after human ingestion over a controlled period of time.

Diffusion Controlled Formulations

The subsequent formulations can be in the form of diffusion controlled formulations which would allow the gradual spread of the subsequent population of particles to scatter or spread out in a liquid medium, are referenced, for example, in U.S. Pat. No. 6,586,006 to Roser et al., which is incorporated by reference herein.

Osmotic Controlled Formulations

Controlled release of the subsequent formulations could be controlled by osmosis. U.S. Pat. No. 6,110,498 to Rudnic et al. for an “osmotic drug delivery system” discloses a system which dispenses a therapeutic agent having limited water solubility in solubilized form. The delivery system comprises a core that is free of swellable polymers and comprises nonswelling solubilizing agents and wicking agents. The solubilized therapeutic agent is delivered through a passageway in the semipermeable coating of the tablet.

U.S. Pat. No. 5,814,979 B2 also to Rudnic et al. describes an osmotic pharmaceutical delivery system comprising (a) a semi-permeable wall that maintains its integrity during pharmaceutical delivery and which has at least one passage therethrough; (b) a single, homogeneous composition within said wall, which composition consists essentially of (I) a pharmaceutically active agent, (ii) at least one non-swelling solubilizing agent which enhances the solubility of the pharmaceutically active agent; (iii) at least one non-swelling osmotic agent and (iv) a non-swelling wicking agent dispersed throughout the composition which enhances the surface area contact of the pharmaceutical agent with the incoming aqueous fluid. Both of these patents to Rudnic et al. are incorporated by reference herein.

Plasma Profile

The plasma profile associated with the administration of a drug compound may be described as a “pulsatile profile” in which pulses of high levetiracetam concentration, interspersed with low concentration troughs, are observed. A pulsatile profile containing two peaks may be described as “bimodal.” Similarly, a composition or a dosage form which produces such a profile upon administration may be said to exhibit “pulsed release” of the levetiracetam.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash-out period provided by the fall off of the plasma concentration of the levetiracetam between peaks has been thought to be a contributing factor in reducing or preventing patient tolerance to various types of drugs.

Because the plasma profile produced by the controlled release composition upon administration is substantially similar to the plasma profile produced by the administration of two or more dosage forms given sequentially, the controlled release composition of the present invention is particularly useful for administering levetiracetam for which patient tolerance may be problematical. This controlled release composition is, therefore, advantageous for reducing or minimizing the development of patient tolerance to the active ingredient in the composition.

In the present invention, the active composition is levetiracetam and the composition in operation delivers the levetiracetam in a bimodal or pulsed manner. Such a composition in operation produces a plasma profile which substantially mimics that obtained by the sequential administration of two IR doses as, for instance, in a typical levetiracetam treatment regime.

The present invention also provides solid oral dosage forms comprising a composition according to the invention. The present invention further provides a method of treating a patient suffering from epilepsy utilizing levetiracetam, comprising administering a therapeutically effective amount of a composition or solid oral dosage form according to the invention to provide pulsed or bimodal administration of the levetiracetam. Advantages of the present invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

Definitions

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “controlled release” as used herein in relation to the composition according to the invention or used in any other context means release of levetiracetam over time and is taken to encompass sustained release and delayed release.

The term “time delay” as used herein refers to the duration of time between administration of the composition and the release of the levetiracetam from a particular component.

The term “lag time” as used herein refers to the time between delivery of active ingredient from one component and the subsequent delivery of levetiracetam from another component.

The active ingredient in each component consists of at least levetiracetam, although a second active ingredient may be desirable for combination therapies. Indeed, two or more active ingredients may be incorporated into the same component when the active ingredients are compatible with each other. A drug compound present in one component of the composition may be accompanied by, for example, an enhancer compound or a sensitizer compound in another component of the composition, in order to modify the bioavailability or therapeutic effect of the drug compound.

Additives

The levetiracetam present in the first and second or subsequent components of the composition may be accompanied by, for example, an enhancer compound or a sensitizer compound in order to modify the bioavailability or the therapeutic effect of the levetiracetam.

As used herein, the term “enhancer” refers to a compound which is capable of enhancing the absorption and/or bioavailability of an active ingredient by promoting net transport across the gastro-intestinal tract in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

Proportion of Levetiracetam and Additives

The proportion of the levetiracetam contained in each component may be the same or different depending on the desired dosing regime. The levetiracetam is present in the first component and in any subsequent component in any amount sufficient to elicit a therapeutic response. The levetiracetam when applicable, may be present either in the form of one substantially optically pure enantiomer or as a mixture, racemic or otherwise, of enantiomers. The levetiracetam is preferably present in a composition in an amount of from 0.1-500 mg, preferably in the amount of from 1-100 mg. Levetiracetam is preferably present in the first component in an amount of from 0.5-60 mg. More preferably, the levetiracetam is present in the first component in an amount of from 2.5-30 mg. The levetiracetam is present in the subsequent components in an amount within a similar range to that described for the first component.

Time Release Profiles

The time release characteristics for the release of the levetiracetam from each of the components may be varied by modifying the composition of each component, including modifying any of the excipients or coatings which may be present. In particular, the release of levetiracetam may be controlled by changing the modified release constituent, including the amount of the modified release coating on the particles, if such a coating is present. As noted above, the time release profiles may be controlled by making the subsequent components or formulations in the form of erodable formulations, diffusion controlled formulations or osmotic controlled formulations. If more than one modified release constituent is present, the modified release coating for each of the subsequent components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be present, in each component, in any amount that is sufficient to yield the desired time lag between components.

The lag time or delay time for the release of the levetiracetam from each component may also be varied by modifying each of the components, including modifying any excipients and coatings which may be present. For example, the first component may be an immediate release component wherein the levetiracetam is released substantially immediately upon administration. Alternatively, the first component may be, for example, a time-delayed immediate release component in which the levetiracetam is released substantially immediately after a time delay. The second component may be, for example, a time-delayed immediate release component as just described or, alternatively, a time-delayed sustained release or extended release component in which the levetiracetam is released in a controlled fashion for up to twenty-four hours.

Plasma Concentration Curve

As will be appreciated by those skilled in the art, the exact nature of the plasma concentration curve will be influenced by the combination of all of these factors just described. In particular, the lag time between the delivery (and thus also the onset of action) of the levetiracetam in each component may be controlled by varying the levetiracetam and coating (if present) of each of the components. Thus, by variation of each component (including the amount and nature of the levetiracetam) and by variation of the lag time, numerous release and plasma profiles may be obtained. Depending on the duration of the lag time between the release of levetiracetam from each component and the nature of the release constituent (i.e., immediate release, sustained release etc.), the pulses in the plasma profile may be well separated and clearly defined peaks (e.g., when the lag time is long) or the pulses may be superimposed to a degree (e.g., when the lag time is short).

In a preferred embodiment, the controlled release composition according to the present invention has a first immediate release component and at least one subsequent or modified release component. The immediate release component comprises a first population of active ingredient containing particles and the modified release components or formulations comprise second or subsequent populations of active ingredient containing particles. The second and subsequent modified release components or formulations may comprise a controlled release coating. Additionally or alternatively, the second and subsequent modified release components or formulations may comprise a modified release matrix material. In operation, administration of such a modified release composition or formulation having, for example, a single modified release component, results in characteristic pulsatile plasma concentration levels of the levetiracetam in which the immediate release constituent of the composition gives rise to a first peak in the plasma profile and the modified release constituent gives rise to a second peak in the plasma profile. Embodiments of the invention comprising more than one modified release constituent give rise to further peaks in the plasma profile.

Such a plasma profile produced from the administration of a single dosage unit is advantageous when it is desirable to deliver two (or more) pulses of active ingredient without the need for administration of two (or more) dosage units. Additionally, in the case of epilepsy it is particularly useful to have such a bimodal plasma profile. For example, a typical levetiracetam treatment regime consists of administration of two doses of an immediate release dosage formulation given four hours apart. This type of regime has been found to be therapeutically effective and is widely used. As previously mentioned, the development of patient tolerance is an adverse effect sometimes associated with levetiracetam treatments. It is believed that the trough in the plasma profile between the two peak plasma concentrations is advantageous in reducing the development of patient tolerance by providing a period of wash-out of the levetiracetam. Drug delivery systems which provide zero order or pseudo zero order delivery of the levetiracetam do not facilitate this wash-out process.

Modified Release Coating Material

Any coating material which modifies the release of the levetiracetam in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit.®. RS and RL, poly acrylic acid and poly acrylate and methacrylate copolymers such as those sold under the Trade Mark Eudragite S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit.®. RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about.5 k-5,000 k), polyvinylpyrrolidone (m. wt. .about.10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about.30 k-300 k), polysaccharides such as agar, acacia, karaya, tragacanth, algins and guar, polyacrylamides, Polyox.®. polyethylene oxides (m. wt. .about. 100 k-5,000 k), AquaKeep.®. acrylate polymers, diesters of polyglucan, crosslinked polyvinyl alcohol and poly N-vinyl-2-pyrrolidone, sodium starch glucolate (e.g., Explotab.®.; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g., Polyox.®., Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g., Eudragit.®., Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

Modified Release Matrix Material

When the subsequent component or formulation comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of the levetiracetam dispersed therein in vitro or in vivo. Modified release matrix materials suitable for the practice of the present invention include but are not limited to microcrystalline cellulose, sodium carboxymethyl-cellulose, hydoxyalkylcelluloses such as hydroxypropylmethyl-cellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixtures thereof.

Form of Dosage

A controlled release composition according to the present invention may be incorporated into any suitable dosage form which facilitates release of the active ingredient in a pulsatile manner. Typically, the dosage form may be a blend of the different populations of levetiracetam for the treatment of epilepsy containing particles which make up the immediate release and the modified release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In this instance the first component of the controlled release composition may be compressed into one layer, with the second component or formulation being subsequently added as a second layer of the multilayer tablet. The populations of levetiracetam containing particles making up the composition of the invention may further be included in rapidly dissolving dosage forms such as an effervescent dosage form or a fast-melt dosage form.

The composition according to the invention preferably comprises at least two populations of levetiracetam particles which have different in vitro dissolution profiles.

Preferably, in operation the composition of the invention and the solid oral dosage forms containing the composition release the levetiracetam in a manner that substantially all of the levetiracetam contained in the first component is released prior to release of the levetiracetam from the second component. When the first component comprises an IR component, for example, it is preferable that release of the levetiracetam from the second or subsequent component is delayed until substantially all the levetiracetam in the IR component has been released. Release of the levetiracetam from the second component may be delayed as detailed above by the use of a modified release coating and/or a modified release matrix material as part of erodable, diffusion controlled or osmotic controlled formulations.

More preferably, when it is desirable to minimize patient tolerance by providing a dosage regime which facilitates wash-out of a first dose of levetiracetam from a patient's system, release of the levetiracetam from the second component or formulation is delayed until substantially all of the levetiracetam contained in the first component has been released, and further delayed until at least a portion of the levetiracetam released from the first component has been cleared from the patient's system. In a preferred embodiment, release of the levetiracetam from the second component of the composition in operation is substantially, if not completely, delayed for a period of at least about two hours after administration of the composition and is released preferably over the remaining twenty-four hour period after administration.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

We claim:

1. A controlled release composition comprising a first component comprising a first population of levetiracetam particles and at least one subsequent component or formulation comprising a subsequent population of levetiracetam particles, wherein the levetiracetam particles contained in the populations are used for the treatment of epilepsy, and the formulation comprising the subsequent population of levetiracetam particles further comprises a modified release constituent comprising a modified release coating, a modified release matrix material, or mixtures thereof, in order that the composition, following oral delivery to a subject, delivers the levetiracetam in the first and subsequent populations in a pulsatile manner.

2. The composition of claim 1, wherein said modified release constituent delivers to a subject the subsequent population over a period of up to twenty-four hours.

3. The composition according to claim 2, comprising a subsequent population for modified and controlled release.

4. The composition according to claim 1, wherein the first population comprises immediate-release particles and the formulation comprising the subsequent population is an erodable formulation.

5. The composition according to claim 1, wherein the formulation comprising the subsequent population is a diffusion controlled formulation.

6. The composition according to claim 1, wherein the formulation comprising the subsequent population is an osmotic controlled formulation.

7. The compositions of claim 1, wherein the formulation comprises a modified release coating.

8. The composition according to claim 7, wherein the composition further comprises an enhancer.

9. The composition according to claim 8, wherein the amount of levetiracetam contained in each of the first and subsequent populations is from about 0.1 mg to about 1 g.

10. The composition according to claim 9, wherein the first and subsequent populations have different in vitro dissolution profiles.

11. The composition according to claim 10, which in operation releases substantially all of the levetiracetam from the first population prior to release of the levetiracetam from the subsequent population.

12. The composition according to claim 11 comprising a blend of the particles of each of the first and subsequent populations contained in a hard gelatin or soft gelatin capsule.

13. The composition according to claim 12, wherein the particles of each of the populations are in the form of mini-tablets and the capsule contains a mixture of the mini-tablets.

14. The composition according to claim 11, in the form of a multilayer tablet comprising a first layer of compressed levetiracetam particles of the first population and another layer of compressed levetiracetam particles of the second population which contain also a second active ingredient.

15. The composition according to claim 14, wherein the first and subsequent populations of levetiracetam-containing particles are provided in a rapidly dissolving dosage form.

16. The composition according to claim 15, wherein the particles of each of the populations are compressed into a fast-melt tablet.

17. A method for the treatment of epilepsy comprising administering a therapeutically effective amount of a composition according to claim 2.

18. The composition according to claim 2, wherein the subsequent formulation comprises a pH-dependent polymer coating which is effective in releasing a pulse of the active ingredient following a time delay.

19. The composition according to claim 18, wherein the polymer coating comprises methacrylate copolymers.

20. The composition according to claim 19, wherein the polymer coating comprises a mixture of methacrylate and ammonio methacrylate copolymers in a ratio sufficient to achieve a pulse of the active ingredient following a time delay.

21. The composition according to claim 20, wherein the ratio of methacrylate to ammonio methacrylate copolymers is 1:1.
